(19) United States
(12) Patent Application Publication
(10) Pub. No.: US 2023/0404105 A1
(43) Pub. Date: Dec. 21, 2023

(54) EDIBLE PROTEIN PRODUCTS

(71) Applicant: ALEPH FARMS LTD., Rehovot (IL)

(72) Inventors: Neta Lavon, Ness Ziona (IL); David Kraus, Rosh Haayin (IL)

(21) Appl. No.: 18/251,219

(22) PCT Filed: Nov. 3, 2021
(86) PCT No.: PCT/IL2021/051304

§ 371 Date: Apr. 28, 2023

Related U.S. Application Data
(60) Provisional application No. 63/108,891, filed on Nov. 3, 2020.

(51) Int. Cl. A23J 3/18; A23J 3/22; C12N 5/077; A23L 33/195; C12M 1/12

(52) U.S. Cl. CPC A23J 3/18; A23J 3/227; C12N 5/0653; A23L 33/195; C12M 25/14; C12N 2533/50

(57) ABSTRACT

The present invention provides edible porous products based on non-animal proteins, which may be used as a protein-based food per se, as well as a scaffold for cell cultures, particularly for the production of cultured food products.

### FIELD OF THE INVENTION

The present invention answers the need for protein-based edible porous products which may be used as a protein-based food per se as well as a scaffold for cell cultures, particularly for the production of cultured food products.

### BACKGROUND OF THE INVENTION

In the past few decades there is an increased interest in food products for human consumption which provide eating experience and nutritional value at least comparable to that of meat, with significantly reduced environmental hazards and without moral issues associated with animal farming. This interest promotes the search for systems, methods and compositions for producing plant-based meat substitutes/analogues as well as cultured meat products (also referred to as cell-based meat, clean meat, cultivated meat and slaughter free meat products).

Plant-based materials used as meat substitutes have been previously described. Among the protein used, wheat gluten has been proposed (see, e.g., Japanese Patent Application Publication No. JPS5332150; U.S. Pat. Nos. 4,938,976 and 6,824,806).

Among others, a challenge in producing meat substitute and clean meat products is the texture and mouth-feel that fail to replicate those of equivalent slaughtered-meat products. Clean meat containing cultured cells only is typically in a form of a ground meat, which significantly limits the variety of food that may be offered to consumers. In addition, commercial scale production of cells in liquid cultures faces problems limiting the available scale. Hybrid products, containing a mixture of cultured cells and plant-based proteins form one potential solution (e.g., Japanese Patent Application Publication No. JPS60118149; U.S. Patent Application Publication No. 20210139843).

Scaffolds have been used in tissue cultivation and engineering for biomedical applications, particularly for implant production or for repair of damaged organs. However, such scaffolds are not suitable for large-scale, cost-effective production of edible products.

International (PCT) Application Publication No. WO/2019/016795 to the Applicant of the present invention disclosed the use of three-dimensional (3D) porous plant-based scaffold for the production of 3D meat-like product. As exemplified therein, textured soy protein (TSP) flakes were used to forms scaffolds having surface area at the mm² range and depth at the μm range.

International (PCT) Application Publication No. WO 2020/106743 discloses methods for generating filamentous organism-based mycelial scaffolds for use in several technologies. In some embodiments, the mycelial scaffold is generated using a perfusion bioreactor system for cell-based meat technologies. The mycelial scaffolds may be generated from a liquid medium or from a solid substrate.

International (PCT) Application Publication No. WO 2020/160533 discloses a cultured meat product comprising a scaffold comprising an electrospun polymer fibre and a population of cells, have a thickness from about 100 mm to about 500 mm. A method of producing the cultured meat product is also disclosed. The cultured meat product may be configured to mimic the taste, texture, size, shape, and/or topography of a traditional slaughtered meat.

International (PCT) Application Publication No. WO 2020/219755 discloses A cultured food product comprising a mixture of myocyte microcarrier scaffold and adipocyte microcarrier scaffold or a heterogeneous scaffold supporting myocytes and adipocytes forming a three-dimensional food product and methods of producing same. However, attempting at production of cultured meat food products, such types of scaffolds have been found not to be satisfactory, particularly in terms of size and stability, for use in cell culture bioreactors for the large-scale production of clean meat products.

The Applicant of the present invention further disclosed a cultivation system for producing cultured food products, particularly cultured meat, on a commercial scale. The cultivation system comprises at least one cell culture bioreactor, typically a plurality of cell culture bioreactors for growing non-human-animal-derived adherent cells on at least one scaffold placed within the cell culture bioreactor (International (PCT) application Publication No. WO 2020/222239).

There is a need and it would be highly useful to have protein-based products that can serve as scaffolds in the industry of meat substitutions and clean meat.

### SUMMARY OF THE INVENTION

The present invention provides protein-based edible porous products having a porous structure, which are highly suitable for use both as a protein-based food per se and as a scaffold for cell cultures, particularly for the production of cultured food products.

In the course of a search for an optimal scaffold to be used with the proprietary cultivation system of the Applicant of the present invention, the inventors have designed hitherto not available protein-based product that answers the needed scaffold characteristics. The products of the invention can be shaped to a variety of shapes and sizes, including planer shapes at sizes suitable for commercial-scale production of cultured food products, particularly cultured meat, not being available hitherto. Furthermore, the designed product can be used per se as a protein-based food or as a component in a variety of foods, including meat substitutes and hybrid meat products.

According to one aspect, the present invention provides an edible product comprising a protein combination and water and having a porous structure, wherein the protein combination comprising at least 40% (w/w) Triticeae gluten out of the total amount of said protein combination and at least one additional type of a non-Triticeae protein, and wherein the porous product is non-extruded.

According to certain embodiments, the Triticeae plant is a Triticum plant. According to certain exemplary embodiments, the Triticum plant is Triticum aestivum (bread wheat). According to certain additional exemplary embodiments, the Triticum plant is Triticum spelta (Spelt).

According to certain embodiments, the protein combination comprises at least 50%, at least 60%, at least 70%, at least 80%, or at least 90% Triticeae gluten out of the total amount of the protein combination. Each possibility represents a separate embodiment of the invention.

According to certain embodiments, the protein combination comprises from about 10% to about 60% of the at least one additional type of non-Triticeae protein.

According to some embodiments, the protein combination comprises between about 70% and 95% wheat gluten out of the total amount of the protein combination. According to certain exemplary embodiments, the protein combination comprises about 90% Triticeae gluten out of the total amount of the protein combination. According to these embodiments the amount of the at least one type of non-Triticeae protein is about 10%.

According to certain embodiments, the at least one additional protein is derived from at least one of a plant other than a Triticeae plant, a fungus, an alga, a single cell microorganism and any combination thereof. Each possibility represents a separate embodiment of the invention.

According to certain embodiments, the single cell microorganism is selected from the group consisting of yeast, microalgae and bacteria. Each possibility represents a separate embodiment of the invention.

According to certain exemplary embodiments, the at least one additional non-Triticeae protein is a plant protein. According to some embodiments, the plant or plant part from which the protein is derived is selected from the group consisting of peas, corn, soybeans, chickpeas, lentils, canola seeds, sunflower seeds, rice, amaranth, lupin, rape-seeds, duckweed, and any combination thereof. Each possibility represents a separate embodiment of the invention.

According to certain embodiments, the at least one additional protein is pea protein.

According to certain embodiments, the at least one additional protein is corn protein (zein).

According to certain embodiments, the at least one additional protein is soy protein.

It is to be explicitly understood that the proteins forming the protein combination are non-extruded proteins.

According to certain embodiments, the amount of the protein combination within the edible porous product is from about 10% to about 50% w/w out of the total product weight.

According to certain embodiments, the amount of the water within the edible porous product is from about 50% to about 90% w/w out of the total product weight. According to certain embodiments, the amount of the water within the edible porous product is from about 50% to about 80% w/w out of the total product weight.

According to certain embodiments, the edible porous product further comprises aqueous soluble salt. Any aqueous soluble salt known to be used in the food industry can be used according to the teachings of the present invention. According to certain exemplary embodiments, the aqueous soluble salt is selected from the group consisting of NaCl, KCl, NaOH, NaCO₃, NaHCO₃ and any combination thereof. Each possibility represents a separate embodiment of the present invention. According to some embodiments, the amount of the salt within the product is from about 0.01% to about 1% w/w out of the total product weight. According to certain exemplary embodiments, the salt is NaCl.

According to certain embodiments, the edible porous product further comprises edible oil and/or fat. According to certain embodiments, the oil or fat is from non-animal source. According to certain exemplary embodiments, the oil and/or fat are in the form of an emulsion.

According to certain embodiments, the edible porous product further comprises at least one agent selected from the group consisting of a flavoring agent precursor, a flavoring agent, a hydrocolloid, a gelling agent, a thickening agent, an emulsifying agent, a binder, a stabilizer, an agent promoting protein-protein bonding, an agent enhancing cell adherence to the edible porous product, an anti-oxidizing agent, a coloring agent and any combination thereof. Each possibility represents a separate embodiment of the present invention.

According to some embodiments, the edible porous product further comprises an agent promoting protein-protein bonding. According to some embodiments, the agent promoting protein-protein bonding is an enzyme. According to certain embodiments, the agent promoting protein-protein bonding is transglutaminase. According to some certain additional or alternative embodiments, the agent promoting protein-protein bonding comprise fibrinogen and/or thrombin. It is to be explicitly understood that the agent promoting protein-protein bonding may be present within the composition in its natural or denatured form, active or inactive.

According to certain exemplary embodiments, the edible product further comprises methyl cellulose.

According to certain embodiments, the edible product has a fibrous texture.

According to certain embodiments, the edible porous product further comprises at least one dietary fiber, at least one amino acid, and a combination thereof.

According to certain embodiments, at least 35% of the total volume of the edible porous product is void volume. The void volume structure of the product is of significance importance when used as a scaffold for cells and/or tissue cultures.

According to certain exemplary embodiments, at least part of the void volume is in the forms of pores. According to certain embodiments, the diameter of the pores is from about 2 μm to about 1.5 mm. According to certain exemplary embodiments, the diameter of the pores is from about 10 μm to about 250 μm. According to certain further exemplary embodiments, the diameter of the pores is from about 10 μm to about 100 μm. The pores within the porous edible material enables liquid passage through the product when subjected to liquid flow.

The porous structure further enables liquid uptake into at least part of the pore volume of said product when subjected to the liquid. According to certain embodiments, subjecting the product to a liquid comprises soaking the product in the liquid. According to certain embodiments, the liquid is an aqueous solution.

According to certain embodiments, the liquid is an aqueous liquid. According to these embodiments, the hydrated product is stable for at least one week.

According to certain embodiments, the edible porous product is characterized by a Young's modulus of from about 10 kPa to about 500 kPa. According to some embodiments, the edible product is characterized by a Young's modulus of from about kPa.

According to certain embodiments, the edible porous product of the invention has a three-dimensional shape selected from the group consisting of a rectangle cuboid, a cylinder and a sphere. Each possibility represents a separate embodiment of the present invention. According to some embodiments, the edible porous product has an uneven three-dimensional shape.

According to certain embodiments, the three-dimension porous product in the shape of a rectangle cuboid comprises at least two surfaces facing opposite directions along and/or in parallel to a longitudinal axis of the rectangle cuboid, wherein none of the surfaces defines an enclosed perimeter across any cross-section thereof.

According to certain exemplary embodiments, the three-dimension porous product in the shape of a rectangle cuboid forms a planar product. According to certain embodiments, the height of the planar product is from 1 mm to 10 cm. According to some embodiments, the height of the planar product is from 1 mm to 5 cm. According to certain embodiments, the height of the planar product is from 1 mm to 1 cm. According to certain exemplary embodiments, the height of the planar product is from 1 mm to 5 mm.

According to certain embodiments, the edible porous product is stable when subjected to cutting pressure. The resulting cuts keep the porous structure and the texture of the source product.

According to certain embodiment, the volume of the three-dimensional shape is from at least about 10 ml and up to about 200,000 ml. According to some embodiments, the volume of the three-dimensional shape is from at least about 10 ml and up to about ml. According to some embodiments, the volume of the three-dimensional shape is from at least about 10 ml and up to about 1,000 ml. According to additional embodiments, the volume of the three-dimensional shape is from at least about 3 ml and up to about 300 ml.

According to certain embodiments, the edible porous product is stable when exposed to a temperature of from about −80° C. to about 250° C.

According to certain embodiments, the edible porous product is for use as a scaffold for culturing at least one type of cells. According to certain exemplary embodiments, the cell are non-human-animal cells. According to certain exemplary embodiments, the scaffold comprising the non-human-animal cells forms a cultured food product, particularly cultured meat product or a part thereof.

According to additional aspect, the present invention provides a cultured food product comprising an edible porous product comprising a combination of proteins and water, wherein the combination of protein comprises at least 40% Triticeae gluten out of the total weight of said protein combination and at least one additional type of non-Triticeae protein; and at least one type of non-human-animal cells.

According to certain embodiments, the Triticeae plant is a Triticum plant. According to certain exemplary embodiments, the Triticum plant is Triticum aestivum (bread wheat). According to certain additional exemplary embodiments, the Triticum plant is Triticum spelta.

The combination of proteins and the protein:water ratio is as described hereinabove.

The types of cells to be comprised within the cultured food would depend on the desired type of food to be produced. According to certain embodiments, the types of cells are selected to produce cultured meat portion that can mimic a cut of slaughtered meat, an offal, or designed for the preparation of a certain dish.

According to certain embodiments, the non-human-animal cells comprise stromal cells and/or endothelial cells and/or fat cells together with at least one cell type according to the desired final meat product, including muscle cells (meat cuts); hepatocytes (liver); cardiomyocytes (heart); renal cells (kidney); lymphoid and epithelial cells (sweetbread made of thymus and pancreas), neural and neuronal cells (brain); ciliated epithelial (tongue) and stomach cells (tripe).

According to certain embodiments, the at least one type of non-human-animal cells is selected from the group consisting of fibroblasts, endothelial cells, fat cells, extracellular matrix (ECM)-secreting cells, muscle cells, progenitor cells thereof and any combination thereof. Each possibility represents a separate embodiment of the present invention.

According to certain exemplary embodiments, the non-human-animal cells are adherent cells.

In some embodiments, at least two or more types of non-human-animal cells are comprised within the cultured food product. According to certain embodiments, the at least two or more cell types are selected from the group consisting of muscle cells, extracellular matrix (ECM)-secreting cells, endothelial cells, fat cells, progenitors thereof, and any combination thereof. Each possibility represents a separate embodiment of the present invention.

The characteristics of the edible porous product, particularly in terms of firmness/softness as reflected by its Young's Modulus values as described herein make the product specifically suitable for the growth of fat cells.

According to certain exemplary embodiments, the present invention provides a cultured food product comprising an edible porous product comprising water and a combination of proteins, wherein the combination of proteins comprises at least 40% Triticeae gluten out of the total weight of said combination and at least one additional type of non-Triticeae protein; and a plurality of non-human-animal fat cells.

According to certain embodiments, the cultured food product comprises cells of a single non-human-animal species origin. In some embodiments, the product comprises cells from a plurality of different species of animals. According to certain embodiments, the non-human-animal is of a species selected from the group consisting of ungulate, poultry, aquatic animals, invertebrate and reptiles. Each possibility represents a separate embodiment of the present invention.

According to certain embodiments, the ungulate is selected from the group consisting of a bovine, an ovine, an equine, a pig, a giraffe, a camel, a deer, a hippopotamus, or a rhinoceros. According to certain exemplary embodiments the ungulate is a bovine. According to certain exemplary embodiments, the bovine is a cow.

In some embodiments, the non-human-animal cells comprise bovine-derived cells selected from extracellular matrix (ECM)-secreting cells, muscle cells, fat cells, endothelial cells, progenitors thereof and combinations thereof. Each possibility represents a separate embodiment of the present invention.

In some embodiments, the bovine-derived cells are bovine pluripotent stem cells (bPSCs). In some embodiments, the bPSCs are embryonic stem cells. In some embodiments, the bPSCs are bovine induced PSCs (biPSCs).

In some embodiments, the bovine-derived cells are cells differentiated from bovine pluripotent stem cells (bPSC). According to certain embodiments, the bovine-derived cells are bovine embryonic fibroblasts (BEFs).

According to certain embodiments, the cultured food product comprises BEFs and/or fat cells differentiated therefrom.

According to certain embodiments, the at least one type of cells is seeded on and/or within the edible porous product. According to certain exemplary embodiments, the at least one type of cells forms a tissue in and/or within the edible porous product. According to these embodiments, the present invention provides an edible porous product comprising a combination of proteins and water, wherein the combination of protein comprises at least 40% Triticeae gluten out of the total weight of said protein combination and at least one additional type of non-Triticeae protein; and at least one type of non-human-animal cells adhered to the edible porous product.

According to certain alternative embodiments, the at least one type of cells is mixed with the edible porous products or parts thereof. According to these embodiments, the cells may or may not be adhered to the edible porous product.

According to further aspect, the present invention provides a method for producing an edible porous product comprising the steps of:
(a) blending a combination of components comprising a proteins combination and water at a ratio of from about 1:1 to about 1:3 protein combination to water, wherein the protein combination comprises at least 40% w/w Triticeae gluten out of the total amount of the protein combination and at least one additional type of non-Triticeae protein to form a malleable material; and
(b) exposing the malleable material to thermal treatment at a temperature of from about 50° C. to about 250° C. at a pressure up to an atmospheric pressure;
thereby producing the edible porous product, said product is characterized by a porous structure.

According to certain embodiments, the Triticeae plant is a Triticum plant. According to certain exemplary embodiments, the Triticum plant is Triticum aestivum (bread wheat). According to certain additional exemplary embodiments, the Triticum plant is Triticum spelta.

According to certain embodiments, the blending step comprises:
(i) mixing the protein combination and potentially at least one additional ingredient to obtain a homogenous blend of dry components;
(ii) adding water and optionally at least one additional liquid to form a mixture;
(iii) kneading or mixing the mixture of dry components, water and optionally at least one additional liquid to form homogenous dough-like material; and
(iv) kneading or mixing the dough-like material of step (iii) until the dough-like material develops gluten network and porous structure.

It is to be explicitly understood that the method of the invention for producing the edible porous product is devoid of extrusion steps.

According to certain embodiments, the kneading or mixing of step (iii) is at a low speed for from about 0.5 min to about 2 min.

According to certain embodiments, the kneading or mixing of step (iv) is at a higher speed compared to the speed applied at step (iii). According to these embodiments, the kneading or mixing is for from about 5 min to about 15 min.

According to certain embodiments, exposing the malleable material to thermal treatment comprises baking said malleable material in an oven pre-heated to a temperature of from about 120° C. about to 150° C. for from about 60 min to about 150 min. According to certain exemplary embodiments, baking is performed within an oven pre-heated to about 140° C. for about 120 min.

According to certain embodiments, exposing the malleable material to thermal treatment comprises placing said malleable material in a bag; removing residual liquids and all gases in the bags using vacuum; and placing the sealed bag in a Sous Vide bath comprising water at a temperature of from about 60° C. about to 100° C. for from about 10 h to about 14 h. According to certain exemplary embodiments, exposing the malleable material to thermal treatment comprises placing said malleable material in a Sous Vide bath comprising water at a temperature of about 80° C. for about 12 h.

According to yet certain additional embodiments, exposing the malleable material to thermal treatment comprises cooking said malleable material in water for from about min to about 2 h, wherein the temperature of the cooking water is kept at from about to about 100° C.

According to yet certain further embodiments, exposing the malleable material to thermal treatment comprises exposing said malleable material to water steam for from about 30 min to about 2 h.

According to some embodiments, the malleable material is wrapped with a packaging material preventing moisture loss before being exposed to the thermal treatment.

According to certain embodiments, the malleable material is shaped before being exposed to the thermal treatment. Any method as is known in the Art for shaping malleable material can be used according the teachings of the present invention. The shapes are as described hereinabove.

According to certain exemplary embodiments, the at least one type of additional protein is derived from organism other than an animal.

According to certain embodiments, the at least one additional type of protein is derived from at least one of a plant other than Triticeae plant, a multi-cellular fungus, an alga, a single cell microorganism and any combination thereof.

According to certain embodiments, the single cell microorganism is selected from the group consisting of yeast, microalgae and bacteria.

According to certain exemplary embodiments, the at least one additional type of protein is a plant protein derived from a plant or a plant part selected from a group consisting of peas, corn, soybeans, chickpeas, lentils, canola seeds, sunflower seeds, rice, amaranth, lupin, rape-seeds, duckweed and any combination thereof.

According to certain exemplary embodiments the additional type of protein is pea protein.

According to certain exemplary embodiments the additional type of protein is corn protein (zein).

According to certain exemplary embodiments the additional type of protein is soy protein.

According to certain embodiments, the combination of components comprises at least one additional ingredient selected from the group consisting of aqueous soluble salt, a flavoring agent precursor, a flavor, a hydrocolloid, a gelling agent, an agent promoting protein-protein bonding, an agent enhancing cell adherence to the edible porous product, anti-oxidizing agent a coloring agent, at least one amino acid, at least one edible fiber and any combination thereof.

According to certain exemplary embodiments, the amount of the aqueous soluble salt is from about 0.01% to about 1% weight/weight out of the total product weight. According to some embodiments, the aqueous soluble salt is selected from the group consisting of NaCl, KCl, NaOH, NaCO₃, NaHCO₃ and any combination thereof.

According to certain embodiments, the method further comprising adding at least one edible oil and/or fat before or after exposure to the thermal treatment.

According to certain exemplary embodiments, the method further comprises forming a three-dimensional shape from the malleable material before exposing said material to the thermal treatment, thereby forming three-dimensional porous product characterized by a porous texture. The three-dimensional shape is typically selected from the group consisting of a rectangle, a cylinder and a sphere.

The properties of the edible porous product produced by the method of the invention are as described hereinabove.

According to some embodiments, the method further comprising a step of seeding at least one type of non-human animal cells on and/or within at least part of the edible porous product.

According to certain embodiments, the method further comprises a step of mixing at least one type of non-human animal cells with the edible porous product or parts thereof.

The non-human-animal cells are as described hereinabove.

According to yet additional aspect, the present invention provides a method for producing a cultured meat product, the method comprising seeding at least one type of non-human animal cells on and/or within at least part of the edible porous product described herein; and culturing the seeded edible porous product under condition enabling the formation of a tissue, thereby producing a cultured meat product.

According to yet a further aspect, the present invention provides a method for producing a composition comprising at least one type of non-human-animal cells and the edible porous product described herein, comprising mixing the at least one type of non-human-animal cells with said edible porous product, thereby producing a hybrid plant-protein-meat product. According to certain embodiments, mixing is performed by adding or seeding said at least one type of cells.

The non-human-animal cells and the edible porous material are as described hereinabove.

According to certain exemplary embodiments, the at least one type of non-human-animal cells are derived from bovine.

According to certain exemplary embodiments, the bovine-derived cells are BEFs.

According to certain additional exemplary embodiments, the bovine-derived cells are fat cells.

According to yet additional aspect, the present invention provides a method for producing a plurality of adipocytes, comprising culturing a plurality of mesoderm-derived non-human animal cells on and/or within an edible porous product comprising a combination of proteins and water, wherein the combination of protein comprises at least 40% Triticeae gluten out of the total weight of said combination and at least one additional type of non-Triticeae protein.

Without wishing to be bound by any specific theory or mechanism of action, the edible porous product described herein, when used as a scaffold for culturing mesoderm-derived cells, particularly bovine-derived mesodermal cells, provide for physical stimuli directing the differentiation of the mesodermal-derived cells to adipocytes. The physical stimuli may replace or reduce the number of otherwise required factors for inducing differentiation of the mesoderm derived cells to adipocytes.

The edible porous product is as described hereinabove.

According to certain embodiments, culturing the plurality of mesoderm-derived non-human-animal cells comprises furnishing said plurality of cells with a cell culture medium.

According to certain embodiments, the non-human animal cells are bovine cell, thereby producing bovine adipocytes.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B show a representative edible material produced. FIG. 1A shows (at the arrow direction) malleable material before baking, after baking, and a cut of the baked product. FIG. 1B shows (at the arrow direction) malleable material subjected to vacuum treatment and baked in a covered baking pan and pressed between 2 baking pans.

FIG. 2 shows average of the pore diameter calculated from four H&E—stained areas of two exemplary scaffolds (G22 and G22 #). Pictures were taken at ×4 magnification.

FIGS. 3A-3B show a representative edible material after exposure to vacuum (FIG. 3A) and after exposure to sous vide heat treatment (FIG. 3B).

FIG. 4. Shows analysis of average Young's modules (kPa) of different samples as indicated.

FIG. 5. Shows Texture Profile Analysis (TPA) of different samples.

FIG. 6. Shows average pore diameter of G70CB and G70CS samples.

FIGS. 7A-7D show histological images of G70CB dry (FIG. 7A), G70CB wet (FIG. 7B), G70CS dry (FIG. 7C), and G70CS Wet (FIG. 7D).

FIG. 8 shows representative images of the samples at the end of the growing phase. Right panel is a picture of G70CB taken an ×2 zoom.

FIGS. 9A-9E show H&E staining of scaffold with cells. The arrows are pointed to the cells. FIGS. 9A and 9B show areas of the scaffold on the first day post seeding. FIGS. 9C and 9D show areas of the scaffold with cells on the 6^{th} day post seeding. FIG. 9E shows a section ×40 magnification of scaffold with cells on the 6^{th} day post seeding. A ×100 magnitude of the scaffold can be seen in the two rectangles on the right.

FIGS. 10A-10D show Picrosirius red (PSR) staining of scaffold with fibroblasts and myoblasts cells. The collagen staining is pointed with arrows. FIG. 10A shows a section ×40 magnification of scaffold with cells on the 6^{th} day post seeding. FIGS. 10B, 10C and 10D show sections ×100 magnification of scaffold with cells on the 6^{th} day post seeding.

FIGS. 11A-11B shows H&E images of G70CS (FIG. 11A) and G70CB (FIG. 11B) with cells.

FIG. 12 shows the edible porous product G70 with seeded cells. Left—G70 baked, right—G70 steamed. Dark grey—scaffolds White/light grey—cells that were seeded and attached to the scaffolds).

FIG. 13 shows a representative picture from a light microscopy of adipocytes differentiated on the edible porous product described herein and washed into the culture medium.

### DETAILED DESCRIPTION OF THE INVENTION

The present invention relates to protein-based edible products having porous structure that is maintained when the products are exposed to a wide range of temperatures and cooking methods, and thus are highly suitable for use as a meat substitute/an analogue product, as a scaffold in the production of cultured food products, particularly cell-based meat products, and as the non-meat protein in hybrid meat products.

The edible porous product of the present invention is advantageous over hitherto known corresponding products at least in that it can be produced at large quantities, and further in its malleable origin that may be shaped to a variety of forms in a variety of sizes making it highly suitable for large-scale, commercial production of a non-meat-protein based food and for cultured meat.

### Definitions

The terms “comprise”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “about” as used herein refers to a variation of a numerical designation of +10% or −10% of the numerical designation.

The terms “animal” and “non-human animal” with reference to cells derived therefrom are used herein interchangeably and refers only to cells of non-human-animals

The term “gluten” as used herein refers to a group of storage proteins that are found in Triticeae plants, including wheat, spelt, barley, rye and more. Triticeae glutens form an elastic network that can trap gas during heating, allowing optimal leavening (for example in breads) and leading to chewy texture in food products. Viscoelastic properties are expressed in wheat (Triticum aestivum) and spelt (Triticum spelta), being exemplary embodiments of the present invention.

As used herein, the term “vital gluten” refers to nondenatured gluten.

As used herein, the term “derived from” with regard to a protein of the present invention refers to a protein isolated from its source. As used herein, the term “isolated” refers to at least partially separated from the natural environment e.g., from a plant cell. According to some embodiments, the term “isolated” refers to a protein in its purified form extracted from the plant or source.

As used herein, the term “scaffold” refers to a three-dimensional structure comprising a biocompatible material that provides a surface suitable for adherence/attachment of cells and the further cultivation (proliferation and/or differentiation and/or maturation) of said cells. A scaffold may further provide mechanical stability and support. A scaffold may be in a particular shape or form so as to influence or delimit a three-dimensional shape or form assumed by a population of proliferating cells. In certain exemplary embodiments, the entire scaffold is made from edible material.

As used herein, the term “food product” refers generally, according to context, to an actual consumable food item or to cultured meat food item that is edible or fit for consumption by an animal without substantial short term and/or long-term adverse effects.

As used herein, the term “cultured meat product” refers to a meat product that is produced by human or machine intervention, rather than grown as a natural component of a living animal. A cultured meat product is thus not obtained directly from the slaughter of a living animal.

According to one aspect, the present invention provides an edible product comprising a protein combination and water and having a porous structure, wherein the protein combination comprises at least 40% (w/w) Triticeae gluten out of the total amounts of the protein combination and at least one additional type of a non-Triticeae protein, and wherein the product is non-extruded.

According to certain embodiments, the Triticeae plant is a Triticum plant. According to certain exemplary embodiments, the Triticum plant is Triticum aestivum (bread wheat). According to certain additional exemplary embodiments, the Triticum plant is Triticum spelta (Spelt).

According to some embodiments, the percentage (w/w) of the Triticeae gluten in the protein combination is at least 45%, at least 50%, at least 55%, at least 60%, at least 65%, at least 70%, at least 75%, at least 80%, at least 85%, at least 90%, or at least 95%.

According to certain exemplary embodiments, the percentage (w/w) of the Triticeae gluten in the protein combination is from about 50% to about 95%, or from about 65% to about 90%. According to certain exemplary embodiments, the percentage (w/w) of the Triticeae gluten in the protein combination is from about 75% to about 95% or from about 80% to about 95%. Each possibility represents a separate embodiment of the present invention. According to certain embodiments, the at least one additional protein is derived from at least one of a plant other than a Triticeae plant, a fungus, an alga, a single cell microorganism and any combination thereof.

According to certain embodiments, the single cell microorganism is selected from the group consisting of yeast, microalgae and bacteria.

According to certain exemplary embodiments, the at least one additional non-Triticeae protein is a plant protein. According to some embodiments, the plant from which the protein is derived is selected from the group consisting of soybeans, corn, peas, chickpeas, lentils, canola seeds, sunflower seeds, rice, amaranth, lupin, rape-seeds, duckweed and any combination thereof.

According to certain currently exemplary embodiments, the protein additional to the Triticeae gluten is pea protein.

According to certain embodiments, the at least one additional protein is corn protein (zein).

According to certain embodiments, the at least one additional protein is soy protein.

According to certain exemplary embodiments, the proteins to be used for the preparation of the protein composition are isolated proteins. According to certain embodiments, each of the protein is a purified protein. The proteins forming the protein combination of the edible porous product described herein are non-textured proteins.

According to certain embodiments, the protein combination comprises from about 10% to about 60% of the at least one additional type of non-Triticeae protein.

According to certain embodiments, the protein combination comprises about 5%, about 10%, about 15%, about 20%, about 25%, about 30%, about 35%, about 40%, about 45%, about 50%, or about 55% of the at least one additional type of non-Triticeae protein.

According to certain embodiments, the percentage (w/w) of the at least one additional type of non-Triticeae protein in the protein combination is from about 5% to about 50%, or from about 10% to about 35%. According to certain exemplary embodiments, the percentage (w/w) of the at least one additional type of non-Triticeae protein in the protein combination is from about 5% to about 35%, or from about 5% to about 20%. Each possibility represents a separate embodiment of the present invention.

According to certain embodiments, the amount of the protein combination within the edible porous product is at least 10%, at least 15%, at least 20%, at least 25%, at least 30%, at least 35%, at least 40%, at least 45%, or at least 50%.

According to certain embodiments, the amount of the protein combination within the edible porous product is from about 10% to about 50% w/w out of the total product weight. According to certain embodiments, the amount of the protein combination within the edible porous product is from about 20% to about 50%, or from about 30% to about 50% or from about 40% to about 50% w/w out of the total product weight.

According to certain embodiments, the amount of the protein combination within the edible porous product is at least 40%.

According to certain embodiments, the amount of the water within the edible porous product is from about 50% to about 90% w/w out of the total product weight.

According to certain exemplary embodiments, the amount of the water within the edible porous product is from about 50% to about 85%, or from about 50% to about 80% w/w out of the total product weight.

According to certain embodiments, the edible porous product further comprises aqueous soluble salt, contributing to the formation of the structure and texture of the product. Any aqueous soluble salt known to be used in the food industry can be used according to the teachings of the present invention. According to certain exemplary embodiments, the aqueous soluble salt is selected from the group consisting of NaCl, KCl, NaOH, NaCO₃, NaHCO₃ and any combination thereof. Each possibility represents a separate embodiment of the present invention. According to some embodiments, the amount of the salt within the product is from about 0.01% to about 1% w/w out of the total product weight. According to some embodiments, the amount is from 0.05% to about 0.5%. According to certain exemplary embodiments, the amount is about 0.1%.

According to certain embodiments, the edible porous food product further comprises an edible oil and/or fat. According to certain exemplary embodiments, the oil/fat is of a non-animal origin. The oil or fat may be comprised within an emulsion, the emulsion form providing for a reduced fat content within the product while maintaining the desired mouth feel attributed to the oil/fat.

According to certain embodiments, the non-animal fat is selected from the group consisting of plant derived oils, algal oils, and oils from bacteria or fungi. According to certain exemplary embodiments, the plant derived oil is selected from the group consisting of coconut oil, mango oil, sunflower oil, cottonseed oil, safflower oil, rice bran oil, cocoa butter, palm kernel oil, palm fruit (kernel) oil, palm oil, soybean oil, rapeseed oil, canola oil, corn oil, sesame oil, walnut oil, almond oil, flaxseed, jojoba oil, castor, grapeseed oil, peanut oil, olive oil, borage oil, algal oil, fungal oil, black currant oil, babassu oil, shea butter, mango butter, wheat germ oil, blackcurrant oil, sea-buckhorn oil, macadamia oil, saw palmetto oil and any combination thereof. Each possibility represents a separate embodiment of the present invention. According to certain embodiments, the non-animal fat is selected from the group consisting of conjugated linoleic oil, arachidonic acid (ARA) enriched oil, docosahexaenoic acid (DHA) enriched oil, eicosapentaenoic acid and (EPA) enriched oil, and any combination thereof. Each possibility represents a separate embodiment of the present invention.

According to certain embodiments, the edible porous product further comprises at least one agent selected from the group consisting of a flavoring precursor, a flavor, a hydrocolloid, a gelling agent, a stabilizing agent, an agent promoting protein-protein bonding, an agent enhancing cell adherence to the edible porous product, an anti-oxidizing agent, a coloring agent and any combination thereof. Each possibility represents a separate embodiment of the present invention.

According to certain embodiments, flavor precursors can be a sugar, a sugar alcohol, a sugar acid, a sugar derivative, an oil, a free fatty acid, an amino acid or derivative thereof, a nucleoside, a nucleotide, a vitamin, an acid, a peptide, a phospholipid, a protein hydrolysate, a yeast extract, or a mixture thereof. Each possibility represents a separate embodiment of the present invention.

According to certain exemplary embodiments, when the scaffold is for production of a cultured meat products, preferred flavor ingredients may include beef flavor, chicken flavor, grill flavor, yeast extract, and any combination thereof. Each possibility represents a separate embodiment of the present invention.

According to certain embodiments, the hydrocolloid is selected from the group consisting of, but not limited to, carboxymethylcellulose, carrageenan, microcrystalline cellulose, xanthan gum, guar gum, konjac, locust bean, pectin, and any combination thereof. Each possibility represents a separate embodiment of the present invention.

According to certain embodiments, the edible porous product further comprises at least one dietary fiber, at least one amino acid, and a combination thereof. The dietary fibers can be soluble or insoluble. According to certain embodiments, the insoluble dietary fibers are selected from Methyl cellulose (E461), Cellulose (E460) and a combination thereof. According to some embodiments, the soluble dietary fibers are selected from guar gum, citrus fibers, vegetable fibers and any combination thereof.

According to certain currently exemplary embodiments the coloring agents are selected from natural coloring agent, for example malt extract or carotenoids.

According to certain embodiments, the edible porous product described herein comprises less than 10% w/w carbohydrates based on the total weight of the product. According to some embodiments, the concentration of carbohydrates within the product is less then 9%, 8%, 7%, 6% or 5% w/w. According to some embodiment's, the edible porous product is essentially devoid of carbohydrates.

According to certain exemplary embodiments, the thickener is methyl cellulose.

The porous structure of the edible porous product of the invention is of significant importance when the product is used as a scaffold for culturing cells and/or tissues and particularly for use in the production of cultured meat. The pore structure, is essential to allow penetration of the cells into the depth of the scaffold and dispersion of the cells to cover the scaffold homogenously. According to certain embodiments, part of the pores is interconnected, typically forming channels, allowing a liquid flow into and through the scaffold and promise the nourishment and growth of seeded cells.

According to certain embodiments, at least 35% of the total volume of the edible porous product comprises pores. The pore diameter may be controlled according to the intended use, during production of the edible porous product. This can be done by controlling the kneading time and speed during kneading the product components. Pore size may be further reduced (if necessary) by exposing the malleable material obtained after kneading to vacuum. Pore size may be further increased (if necessary) by adding pore enhancing materials and/or materials that release gaseous substance during the process, for example bicarbonates (including sodium bi-carbonate, ammonium bi-carbonate) hydrogen peroxide, and others. The temperature to which the malleable material is then exposed further affects the pore diameter. According to certain embodiments, the diameter of the pores is from about 2 μm to about 1.5 mm. According to certain exemplary embodiments, the diameter of the pores is from about 5 μm to about 250 μm. According to certain further exemplary embodiments, the diameter of the pores is from 5 or about 10 μm to about 50 μm, or about 60 μm, or about 70 μm, or about 80 μm, or about 90 μm or about 100 μm, or about 110 μm, or about 120 μm, or about 130 μm, or about 140 μm or about 150 μm.

The porous structure of the edible porous product enables both liquid flow through the product void volume and liquid capture within the void volume when the product is soaked in a liquid.

According to certain embodiments, the pores of the porous product are distributed along various dimensions of the porous products. The pores can be evenly or not-evenly distributed. According to certain embodiments, the edible porous product comprises pores in a form of channels enabling flow of a liquid through the porous product. The flow rate of a liquid passing through the product pores may be adapted according to the intended use of the porous product and the intended function of the liquid. The type of the liquid will also depend on the intended use. For example, when the porous product is used as a scaffold for culturing cells, the liquid can be an aqueous buffer for washing the product, a cell suspension, or a culture medium for supporting the growth of the cultured cells. When the liquid is, for example, a culture medium, the flow rate may be adapted to nourish the cells present throughout the scaffold.

According to certain embodiments, when the liquid is an aqueous liquid, the hydrated product is stable for at least one week. As used herein, a “stable” product refers to a product of the invention preserving its structure and/or shape and/or texture.

According to certain embodiments, the edible porous product of the invention is characterized by a Young's modulus of from about 30 kPa to about 150 kPa. The Young's Modulus is a measure of the stiffness of the product. According to certain exemplary embodiments, the Young's Modulus is measured by Texture Profile Analysis (TPA). TPA can be measured by any method as is known in the art. For example, TPA can be measured by double compression test of the edible porous product. According to certain embodiments, the Young's modulus of the edible porous product described herein is from about 30 kPa to about 80 kPa. According to some embodiments, the Young's modulus of the edible porous product described herein is from about 40 kPa to about 70 kPa. According to yet certain additional embodiments, the Young's modulus of the edible porous product described herein is from about 80 kPa to about 150 kPa. According to some embodiments, the Young's modulus of the edible porous product described herein is from about 90 kPa to about 120 kPa.

According to certain embodiments, the porous structure of the product of the present invention enables liquid flow through the pores of said product when subjected to the liquid.

According to certain embodiments, the edible porous product of the invention has three-dimensional shape selected from the group consisting of a rectangle cuboid, a cylinder and a sphere. Each possibility represents a separate embodiment of the present invention. According to some embodiments, the edible porous product of the invention has a three-dimensional irregular shape. The shape and the volume of the product is preferably set during its production according to the intended use.

According to certain exemplary embodiments, the edible porous product is shaped to a regular three-dimension shape when used as a scaffold for cells and/or tissue cultured.

According to certain alternative exemplary embodiments, the edible porous product is shaped to a an irregular three-dimension multiple shapes when used as a component in a hybrid mixture of non-animal-protein and animal cells and/or tissues.

Advantageously, the product of the present invention can be shaped at a wide range of volumes, from about 1 ml and up to 200 liters. Accordingly, when used as a scaffold for seeding cells, and particularly as a scaffold for culturing nom-human-animal cells to produce cultured meat, the scaffold shape and volume can be adapted according to the design of the cultivation system. It is to be explicitly understood that scaffolds according to the teachings of the invention can be used with any cell culture bioreactor and/or cultivation system known in the art. An exemplary cultivation system in which the scaffolds of the invention can be used are described in WO 2020/222239 to the Applicant of the present invention.

According to certain exemplary embodiments, when designed as a scaffold for producing cultured meat, the product volume is in the range of 500 ml to 10 liters.

Additional advantage of the edible porous product of the invention is its stability upon exposure to heat. This feature is particularly important when the product forms part of a cultured meat food product. The stability of the product in a temperature range of from about −80° C. to about 250° C. enables its storage at low temperature (−80° C. to 0° C.) for a duration of up to 12 months; sous vide cooking (50-95° C.), oven cooking (120-250° C.), pan/oil frying (130-260° C.), and grilling (200-300° C.).

According to certain exemplary embodiments, the edible porous product of the invention is used as a scaffold suitable for growth of non-human animal cells, particularly for the production of cultured meat food products. Any non-human animal cells and any method of seeding and cultivating the cells known in the art for the production of cultured meat food can be used with the edible porous scaffolds of the invention. According to certain exemplary embodiments, the non-human-animal cells are disclosed in International (PCT) Application Publication No. WO 2019/016795.

The present invention explicitly encompasses a cultured meat product comprising at least one type of non-human-animal cells attached to the edible porous product of the invention.

To produce cultured meat, two or more types of non-human-animal cells, particularly adherent cells are selected according to the desired type of meat portion to be produced. The produced meat portion can mimic a cut of slaughtered meat, an offal, or designed for the preparation of a certain dish.

According to certain embodiments, the non-human-animal cells comprise stromal and/or endothelial cells and/or fat cells together with at least one cell type according to the desired final meat product, including muscle cells (meat cuts); hepatocytes (liver); cardiomyocytes (heart); renal cells (kidney); lymphoid and epithelial cells (sweetbread made of thymus and pancreas), neural and neuronal cells (brain); ciliated epithelial (tongue) and stomach cells (tripe).

According to certain embodiments, the non-human-animal cells are selected from the group consisting of muscle cells, extracellular matrix (ECM)-secreting cells, fat cells, endothelial cells, and progenitors thereof. In some embodiments, the two or more types of non-human-animal cells comprise muscle cells or progenitors thereof and at least one additional type selected from the group consisting of ECM-secreting cells, fat cells, endothelial cells, and progenitors thereof. In some embodiments, the non-human-animal cells comprise muscle cells or progenitors thereof, ECM-secreting cells or progenitors thereof, fat cells or progenitors thereof, and endothelial cells or progenitors thereof.

According to certain embodiments, the non-human-animal is selected from the group consisting of ungulate, poultry, aquatic animals, invertebrate and reptiles. Each possibility represents a separate embodiment of the present invention.

According to certain embodiments, the ungulate is selected from the group consisting of a bovine, an ovine, an equine, a pig, a giraffe, a camel, a deer, a hippopotamus, or a rhinoceros. According to some embodiments the ungulate is a bovine. According to certain exemplary embodiments, the bovine is a cow.

In some embodiments, the non-human-animal derived cells to be seeded on the edible porous scaffold of the present invention comprise pluripotent stem cells. According to certain embodiments, the non-human-animal derived cells to be seeded comprise bovine-derived pluripotent stem cells (bPSCs). According to certain embodiments, the bPSCs are bovine embryonic stem cells. According to certain embodiments, the bPSCs are bovine induced pluripotent stem cells (biPSCs). The seeded bovine-derived cells are grown under conditions enabling differentiation to the desired cell types. In some particular embodiments, the seeded pluripotent bovine-derived cells are differentiated to muscle cells, ECM-secreting cells, fat cells and/or endothelial cells.

In some embodiments, the non-human-animal derived cells to be seeded on the edible porous scaffold of the present invention comprise differentiated cells.

According to additional aspect, the present invention provides a method for producing an edible porous product comprising the steps of:
(a) blending a combination of components comprising a proteins combination and water at a ratio of from about 1:1 to about 1:3 protein combination to water, wherein the protein combination comprises at least 40% w/w Triticeae gluten out of the total amount of the protein combination and at least one additional type of non-Triticeae protein to form a malleable material; and
(b) exposing the malleable material to thermal treatment at a temperature of from about 50° C. to about 250° C. at a pressure up to an atmospheric pressure;
thereby producing the edible porous product, said product is characterized by a porous structure.

According to certain embodiments, the Triticeae plant is a Triticum plant. According to certain exemplary embodiments, the Triticum plant is Triticum aestivum (bread wheat). According to certain additional exemplary embodiments, the Triticum plant is Triticum spelta.

According to certain embodiments, the blending step comprises:
(i) mixing the protein combination and potentially at least one additional ingredient to obtain a homogenous blend of dry components;
(ii) adding water and optionally additional liquids to form a mixture;
(iii) kneading or mixing the mixture of dry components, water and optionally at least one additional liquid to form homogenous dough-like material; and
(iv) kneading or mixing the dough-like material of step (iii) until the dough-like material develops gluten network and porous structure.

It is to be explicitly understood that the method of the invention for producing the edible porous product is devoid of extrusion steps.

According to certain embodiments, the kneading or mixing of step (iii) is at a low speed for from about 0.5 min to about 2 min.

According to certain embodiments, the kneading or mixing of step (iv) is at a higher speed compared to the speed applied at step (iii). According to these embodiments, the kneading or mixing is for from about 5 min to about 15 min.

According to certain embodiments, the thermal treatment comprises sous vide treatment. According to theses embodiments, the malleable material is placed in a bag, and residual liquid and all gases in the bags are removed using vacuum; and the sealed bag is placed in a Sous Vide bath comprising water at a temperature of from about 60° C. about to 100° C. for from about 10 h to about 14 h. or at a temperature of about 80° C. for about 12 h. According to certain exemplary embodiments, the vacuum is applied at a pressure of from (−)1 to zero bars.

According to certain additional or alternative embodiments, the thermal treatment comprises baking. According to theses embodiments, the baking temperature is from about 120° C. to about 250° C. According to certain exemplary embodiments, backing comprises placing the malleable material in an oven pre-heated to a temperature of from about 120° C. about to 150° C. for from about 60 min to about 150 min. According to certain further exemplary embodiments, baking is performed within an oven pre-heated to about 140° C. for about 120 min.

According to certain additional or alternative embodiments, the thermal treatment comprises cooking. According to certain exemplary embodiments, cooking is performed by placing said malleable material in water for from about 30 min to about 2 h, wherein the temperature of the cooking water is kept at from about 80° C. to about 100° C.

According to certain additional or alternative embodiments, the thermal treatment comprises steaming. According certain embodiments, steaming is performed using water steam for from about 30 min to about 2 h.

According to some embodiments, the malleable material is wrapped with a packaging material preventing moisture loss before being exposed to the thermal treatment. The packaging material can be any known food-grade material resistant at the desired temperature, including, for example, baking paper and aluminum foil.

According to certain embodiments, the malleable material is shaped before being exposed to the thermal treatment. Shaping may be performed using dough shape machine or equipment (see, e.g., rondo-online.com/en). The shapes are as described hereinabove.

According to certain embodiments, the method further comprises placing the malleable material under ambient conditions for 90 minutes to reach Load at maximum load of 0.4 to about 2.0 N. According to certain exemplary embodiments, the Load is detected by a tensile test using Texture analyzer.

According to yet additional aspect, the present invention provides a method for producing a cultured meat product, the method comprising seeding at least one type of non-human-animal cells on and/or within at least part the edible porous product of the present invention. The edible porous product and the cell types to be used for producing the clean meat product are as described hereinabove.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Production of Edible Porous Product with Baking as Heat Treatment

TABLE 1
ingredients and method used for producing porous edible products

| | | | | Guar | | | |
|---|---|---|---|---|---|---|---|
| | | | | Gum | | Cold | |
| | Vital | | | (Shree | Salt | Water | |
| | Wheat | Pea Protein | Citrus | Ram | (Salt | Purified | |
| | Gluten | Concentrate | Fibre | India | of the | by | |
| Recipe | (Roquette) | (Cargill) | (Ceamsa) | Gums) | earth) | Tami4 ® | |
| Tag | (g) | (g) | (g) | (g) | (g) | (g) | Vacuum |
| G22 | 250 | 50 | 2.5 | 0.5 | 1 | 600 | No vacuum |
| G22^{#} | 250 | 50 | 2.5 | 0.5 | 1 | 600 | Vacuum |
| | | | | | | | before |
| | | | | | | | thermal |
| | | | | | | | treatment |
| | | | | | | | (steps 8-9) |

### Equipment

Glass beakers (1000 ml)
N50 Kneader by Hobart
Vacuum machine—SQUARE 400-B, INTER-COM
Vacuum bags
Toaster Oven by Morphy Richards
Baking pans
Parchment paper
Refrigerator—BAMBAS frost
Zip-lock bags (Large)
Tami4 ®—MAZE Water Purification System cleaning water of organic and inorganic toxic residues as well as bacteria and viruses using filters and UV.

### Method

1. Weigh all dry ingredients in separated small bowls and mix together in the kneader bowl (e.g., N50 Kneader by Hobart) until homogeneous (for example: 1-minute, speed #1).
2. Weigh the wet ingredients (including water and additional wet ingredients like colouring agents, oils etc., if required) in a 1000 ml glass beaker and mix until homogeneous.
3. Add the wet ingredients to the kneader bowl with the dry ingredients.
4. Knead/blend/mix in a kneader (e.g., Hobart N50) using the “J” hook: 2 minutes on slow speed (for example: speed #1); 8 minutes on medium speed (for example: speed #2).
5. Knead until an even dough-like substance is obtained that is pliable and somewhat sticky.
6. Shape to a flat rectangular shape (for example: 20 cm width×15 cm length×3 cm height).
7. Place the shaped material into a baking pan lined with parchment paper.
8. Optionally, insert the shaped material inside a large vacuum bag, and run Vacuum machine (e.g., SQUARE 500 230V 50 HZ, Busch) on program number 3.
9. Take out of the vacuum bag and drain extra fluids if needed.
10. Put in a pre-heated oven (e.g., Toaster Oven by Morphy Richards set to turbo, 140° C., for 120 minutes.
11. Take out of the baking pan and cool to room temperature (about 20° C.).
12. Keep in a Zip lock bag, at 4° C.

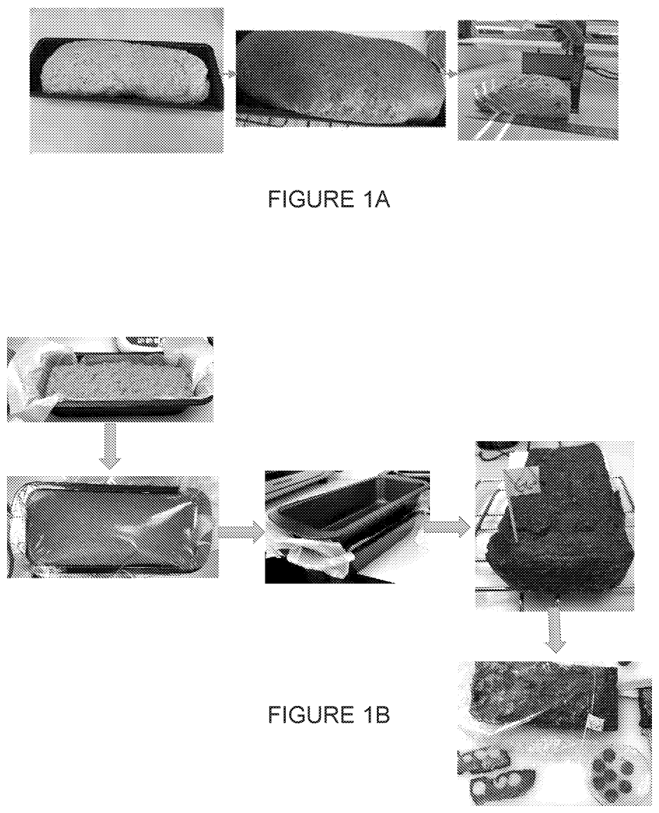

FIG. 1 shows an exemplary product produced by the method described above without (FIG. 1A) or with (FIG. 1B) vacuum treatment before exposure to heat treatment.

### Example 2: Pore Analysis

Pore analysis and in particular the average pore diameter provides an indication of the empty spaces volume and the channels formed within the composition. When the composition is used as a scaffold for cell cultivation, the empty spaces and channels allow the flow of cultivation medium as to nourish and support the growth of cells seeded on the scaffold which also penetrate into the empty spaces.

Each of the edible products was prepared separately, according to a recipe described in Example 1 hereinabove.

The products were cut into 1×1×1 cm³ cubes, fixed in 4% paraformaldehyde (PFA), and thereafter subjected to H&E staining, which differentially stains positive and negative charges of the composition components. Gray area indicates a stained composition and white area indicates space (void volume). The void volume was calculated using ImageJ, a Java-based image processing program developed at the National Institutes of Health and the Laboratory for Optical and Computational Instrumentation (LOCI, University of Wisconsin). The pore diameter was measured manually.

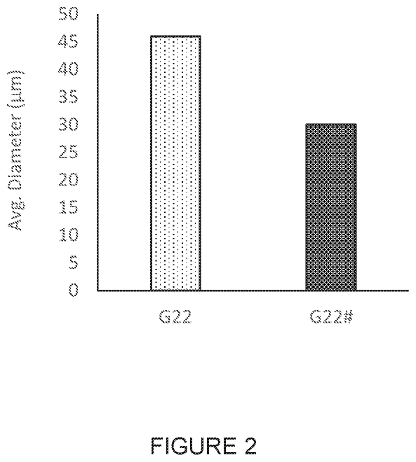

Four captures of each image were analyzed using ImageJ software analysis tool (Analyze particles). Table 2 below shows the pore analyses for the two samples. FIG. 2 shows average of the pore diameter in a scaffold cut (×4 magnification), calculated from four sections in the cut.

TABLE 2
Pore analyses results

| | G22 | G22^{#} |
|---|---|---|
| Number of pores/Image | 4813.8 | 7436.0 |
| Avg. void volume square root of the | 40.8 | 26.5 |
| (μm) | | |
| Smallest void volume square root (μm) | 2.2 | 2.2 |
| Largest void volume square root (μm) | 1434.1 | 1091.7 |
| Avg. Diameter* (μm) | 46.0 | 30.0 |

G22^{#} is prepared using the same recipe and method as G22, with the exception of vacuum before heat treatment (steps #8-9).
*Diameter was calculated by the following formula:
$D = \frac{\sqrt{4 A}}{π}$ Wherein A = Average Area of the pores and π = 3.14159

Wherein A=Average Area of the pores and π=3.14159

### Example 3: Production of Edible Porous Product with Sous Vide as Heat Treatment

TABLE 3
ingredients of porous edible product G56

| | | | Guar | | | Caramel | | |
|---|---|---|---|---|---|---|---|---|
| | | | Gum | | | Powder | | |
| | Vital | | (Shree | Salt | | 600 (DD | | |
| | Wheat | Pea Protein | Ram | (Salt | Cold | Williamson | | |
| | Gluten | Concentrate | India | of the | Water | food | Canola oil | Soy sauce |
| Recipe | (Roquette) | (Cargill) | Gums) | earth) | (Tami4) | colors) | (Etzhazait) | (Kikkoman) |
| Tag | (g) | (g) | (g) | (g) | (g) | (g) | (gr) | (gr) |
| G56 | 250 | 25 | 0.5 | 1 | 594 | 3 | 46 | 6 |

### Equipment

Glass beakers (1000 ml)
N50 Kneader by Hobart
Vacuum machine—SQUARE 500 230V 50 HZ, Busch
Sous vide—ANOVA
Vacuum bags
Refrigerator—BAMBAS frost
Plastic dough scraper
Ice pack (reusable)

### Method

1. Weigh all dry ingredients in separated small bowls and mix together in the kneader bowl (e.g., N50 Kneader by Hobart) until homogeneous (for example: 1-minute speed #1).
2. Weigh the wet ingredients (including water and additional wet ingredients like colouring agents, oils etc., if required) in a 1000 ml glass beaker and mix until homogeneous.
3. Add the wet ingredients to the kneader bowl with the dry ingredients.
4. Knead/mix/blend in a kneader (e.g., Hobart N50) using the “J” hook: 2 minutes on slow speed (for example: speed #1); 8 minutes on medium speed (for example: speed #2).
5. Knead until an even dough-like substance is obtained that is pliable and somewhat sticky.
6. Shape to a flat rectangular shape (for example: 20 cm width×15 cm length×3 cm height).
7. Drain the excess water if needed and place in a vacuum bag.
8. Run Vacuum machine on program number 3.
9. Push the mass into a rectangle using a dough scraper.
10. Put the sealed vacuum bag into the preheated Sous vide (80° C.) and cook over night.
11. Transfer the bag directly into ice cold water (tap water with reusable ice-packs).
12. Keep at 4° C.

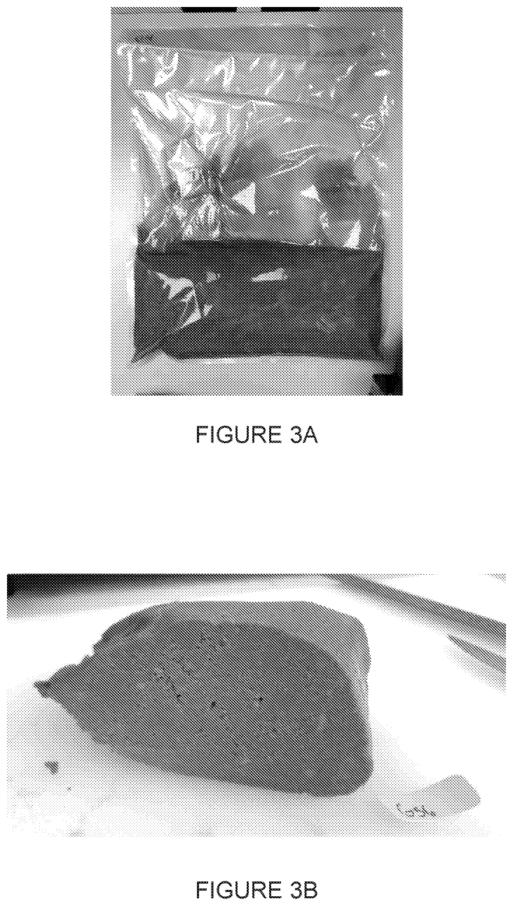

FIG. 3 shows exemplary porous product in a vacuum bag before exposure to sous vide heat treatment (FIG. 3A) and after sous vide heat treatment (FIG. 3B).

The product obtained using sous vide as the heating treatment is less porous, denser and firmer compared to the product obtained by baking as described in Example 1 hereinabove. Its organoleptic properties are also different, having a rubbery and less spongy texture. The different characteristics may be attributed to the vacuum applied and to the sous vide heating conditions.

### Example 4: Production of Edible Porous Products by Several Recipes and Cooking Methods

TABLE 4
ingredients used for producing porous edible products

| | | | | Isolated | | | Methyl- |
|---|---|---|---|---|---|---|---|
| | Vital | Pea Protein | Zein | Soy | | Cold water | Cellulose |
| | Wheat | Concentrate | (Corn | Protein | | Purified by | (Tylopur |
| Recipe | Gluten | (PPC) | protein) | (ISP) | Salt | Tami4 ® | MCE-100TS) |
| Tag | (gr) | (gr) | (gr) | (gr) | (gr) | (gr) | (gr) |
| G68 | 750 | — | 75 | — | 15 | 1500 | — |
| G69 | 750 | — | — | 75 | 15 | 1500 | — |
| G70 | 750 | 75 | — | | 15 | 1500 | — |
| G72 | 750 | 75 | — | | 15 | 1500 | 38 |

### Equipment:

Glass beakers (1000 ml)
Quart Mixer—HL-11012 (AT)
Slicing machine—NOAW
Stainless steel Steamer
Toaster Oven—Morphy Richards
Baking tray (300×220 mm)
Non-stick silicon baking mat (Silpat)
Rolling pin
Pan
Parchment paper
Aluminum foil
Refrigerator—BAMBAS frost
Zip-lock bags (Large)

### For G68-G70:

1. Weigh all dry ingredients in separated small bowls and mix together in the kneader bowl (e.g., Quart Mixer—HL-11012 (AT)) until homogeneous (for example: 1-minute, speed #1).
2. Weigh the wet ingredients (including water and additional wet ingredients like coloring agents, oils etc., if required) in a 1000 ml glass beaker and mix until homogeneous.
3. Add the wet ingredients to the kneader bowl with the dry ingredients.
4. Knead/blend/mix in a kneader (e.g., Quart Mixer—HL-11012 (AT)) using the “J” hook: 2 minutes on slow speed (for example: speed #1); 8 minutes on medium speed (for example: speed #2).
5. Knead until an even dough-like substance is obtained that is pliable and somewhat sticky.
6. For recipe G72 only: insert the mass into a medium size bowl covered with a plastic wrap and place in the refrigerator until the mixture reaches a temperature below 5° C. (12-24 hours). The incubation in low temperature is required for activation of the added thickener (methyl cellulose [Tylopur® MCE]).
7. Continue to one of the cooking methods procedures:

### Covered Baking (CB):

1. Weigh 300 g of the mass and shape into a rectangle 297×210×7 mm (A4 size) on a baking tray, lined with non-stick silicon baking mat (Silpat).
2. Let the dough rest for 20 minutes at room temperature (RT)—gluten relaxation.
3. Place another non-stick silicon baking mat and use a rolling pin to spread the dough, to ensure total coverage of the baking tray.
4. Keep the baking mat over of the dough and place additional baking tray on top to prevent browning and over drying.
5. Bake in a pre-heated oven—turbo, 140° C., for 60 minutes.
6. Take out of the baking pan and cool to room temperature (about 20° C.).

### Covered Cooking (CC):

1. Weigh 300 g of the mass and shape into a rectangle 297×210×10 mm (A4 size).
2. Heat water in a pan, keep it at low heat (80-100° C.).
3. Cover the rectangle with parchment paper.
4. Cover with additional layer of aluminum foil.
5. Insert the shaped rectangle inside the pan and close the lid.
6. Cook it for 45 minutes.
7. Remove the rectangle from the pan (keep it covered).
8. Keep at 4° C. in a big Zip lock bag.

### Steaming (S):

1. Weigh 300 g of the mass and shape into a rectangle 297×210×10 mm (A4 size).
2. Boil water in a steamer, keep it at low heat.
3. Insert the rectangle inside the steamer and close the lid.
4. Cook it for 45 minutes.
5. Remove the rectangle carefully (keep it covered).
6. Keep at 4° C. in a big Zip lock bag.

### Covered Steaming (CS):

1. Weigh 500 g of the mass and place it in between two baking sheets.
2. Let the dough rest for 20 minutes, RT—gluten relaxation.
3. Shape into a rectangle 297×210×7 mm (A4 size) by using a rolling pin over the two baking sheets.
4. Insert the rectangle into an oven roasting bag and close it with a tie.
5. Boil water in a steamer, keep it at low heat (80-100° C.).
6. Place the bag in the steamer and close the lid.
7. Cook it for 45 minutes.

### Slicing to 3 mm Thickness:

1. Slice an external layer to remove the crust—use a slicing machine (e.g., Slicing machine—NOAW, thickness control knob=2).
2. Use the slicing machine to slice 3 mm thick samples, thickness control knob=3.
3. Keep at 4° C. in a Zip lock bag

### Example 5: Texture Profile Analysis (TPA) Compression Test of the Edible Porous Product

TPA test provides an indication of the product's mechanical properties, with Young's Modulus being the main parameter to compare the firmness/softness of composition obtained by different methods. The higher the value of Young Modulus is, the product is stiffer. The stiffness of the product provides for its organoleptic properties in terms of tough/hard bite.

The TPA compression test was performed as follows:

Texture Profile Analysis (TPA) is a double compression test under constant load, determining the textural properties of foods. Young's Modulus is the measure of stiffness of the sample and it is determined as the slope within the linear region of the stress-strain curve of the first compression.

Young's modulus of products having the G70 recipe (Table 4) and produced by methods described above was determined. The products were sliced into 3-5 mm thick slices. The samples were placed in the Texture analyser (LLOYD). TPA double compression test was performed, using round, cylindrical “Magnus” probe and the Young's modulus (as part of the TPA test) was calculated by NexygenPlus 3 Materials Testing Software.

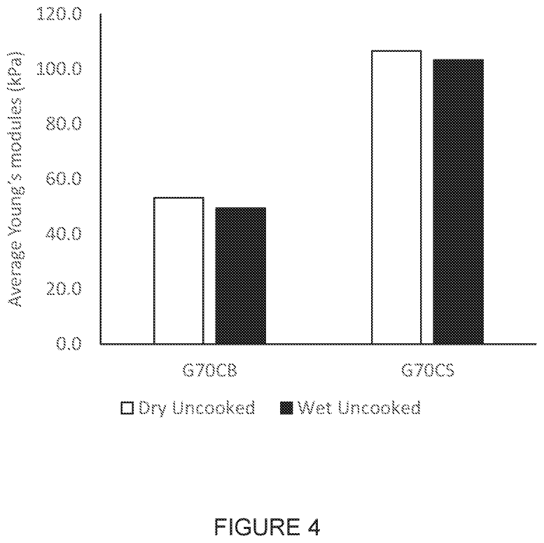

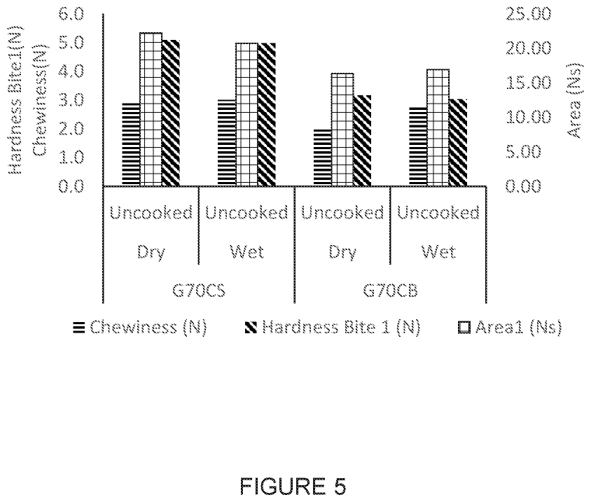

Average Young's modules (kPa) are shown in FIGS. 4 and Table 5, and TPA results and are shown in FIG. 5.

Analysis of the pore diameter of the product having G70 recipe performed as described in Example 2 hereinabove are presented in Table 6 below.

TABLE 5
Young's modulus values

| | | Average | | |
|---|---|---|---|---|
| | | Young's | | |
| | | Modulus | | |
| Sample Name | Conditions | (kPa) | SD | SE |
| G70 Covered Baked (3 mm) | Dry | 53 | 5 | 2.6 |
| G70 Covered Steamed (3 mm) | Dry | 106 | 11 | 5.7 |
| G70 Covered Baked (3 mm) | Wet | 49 | 6 | 3.0 |
| G70 Covered Steamed (3 mm) | Wet | 103 | 20 | 6.9 |

*“Dry” product refers to the product obtained after baking. “Wet” product refers to the product obtained after baking further hydrated in saline for at least 4 nights (about 84 h)
Average Young's modules (kPa) and TPA results are shown in FIGS. 4 and 5, respectively.
Analysis of the pore diameter of the product having G70 recipe and produced as detailed in Table 6 below was performed as described in Example 2 hereinabove.

TABLE 6
Pore diameter of G70 products produced as indicated

| G70 CB Dry | | | G70 CB Wet | | |
|---|---|---|---|---|---|
| | Void Volume | | | Void Volume | |
| | Square Root | Diameter | | Square Root | Diameter |
| | (μm) | (μm) | | (μm) | (μm) |
| Mean | 1299.0 | 40.7 | Mean | 675.1 | 29.3 |
| SD | 28145.0 | | SD | 23373.3 | |
| SE | 128.8 | | | 87.2 | |
| Min | 4.6 | 2.4 | Min | 4.6 | 2.4 |
| Max | 2373652.4 | 1738.5 | Max | 3103503.3 | 1987.8 |
| 70CS Dry | | | G70CS Wet | | |
| | Void Volume | | | Void Volume | |
| | Square Root | Diameter | | Square Root | Diameter |
| | (μm) | (μm) | | (μm) | (μm) |
| Mean | 432.6 | 23.5 | Mean | 967.4 | 35.1 |
| SD | 12990.7 | | SD | 14217.0 | |
| SE | 49.2 | | SE | 81.3 | |
| Min | 4.6 | 2.4 | Min | 4.6 | 2.4 |
| Max | 1846571.9 | 1533.3 | Max | 1070630.1 | 1167.5 |

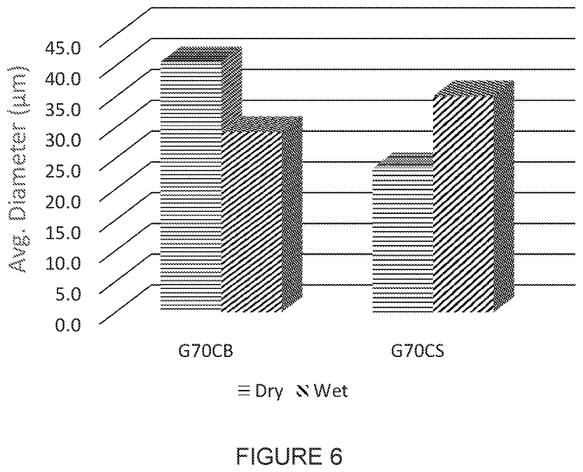

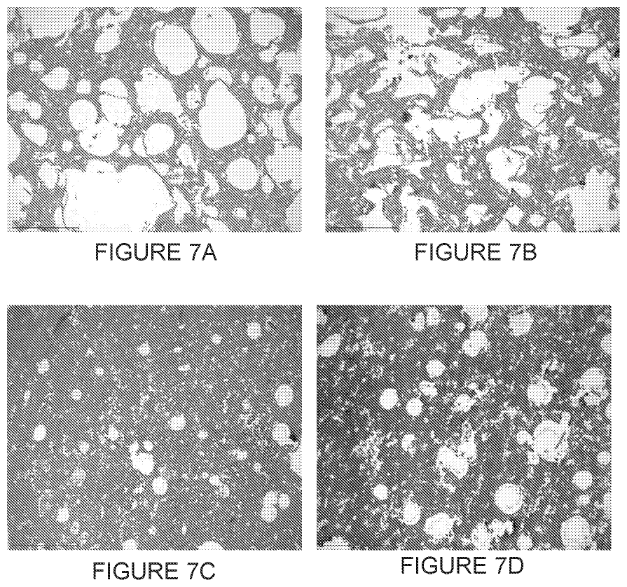

The average pore size is shown in FIG. 6. Histological images of the products are shown in FIGS. 7A-7D.

### Example 6: Bovine Cells Adherence and Growth on a Scaffold of the Invention

To enhance the attachment of the non-human-animal cells to the plant-based scaffold without special coating, the scaffold (G70CB and G70CS) was pretreated with ethanol in order to create a hydrophilic surface enhancing the cells adherence.

The scaffold was treated with gradient ethanol solutions of ethanol:water v/v of 80%, 60%, 40% and 20%. The scaffold was incubated with each ethanol solution for 1 hour at room temperature. The scaffold was initially soaked in 80% ethanol solution and then transferred to the next solutions of 60%, then 40% and finally 20%.

The scaffold was washed three times with dH₂O and then with Phosphate-buffered saline (PBS) until the osmolality was below 290 mOsm/kg, as of PBS.

The scaffold was inoculated with bovine fibroblasts and myoblasts and grown for subsequent 6 days under static incubation at a temperature of 38.5° C. and 5% CO₂.

Samples from the scaffold were taken for staining on day 1 after seeding and at the end of the experiment on the 6^{th} day. The slides stained by H&E (hematoxylin and eosin staining, staining nuclei in blue and extracellular matrix and cytoplasm in pink) and PSR (Picrosirius red, staining collagen in red and cytoplasm in yellow).

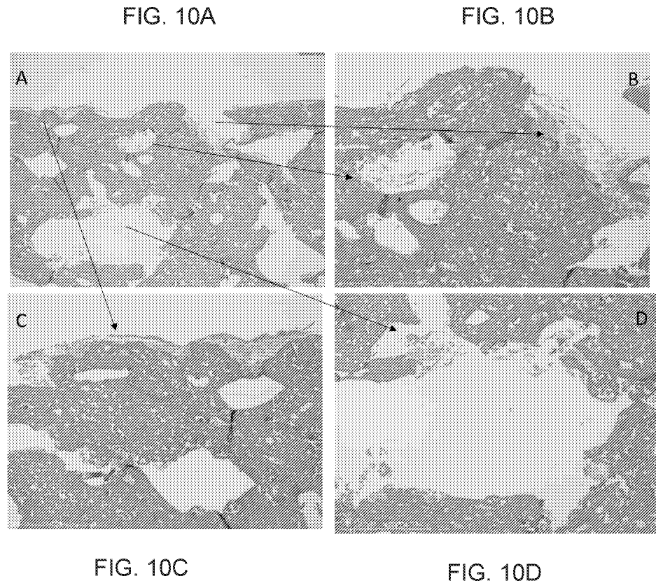

It can be clearly observed in FIG. 10 that the cells have been adhered to the scaffold as depicted in images taken at day 1 (FIGS. 10A-10B) and that the cells further proliferated on the scaffold during the 6 days of incubation (FIGS. 10C-10D). The cell mass on the 6^{th} day post seeding was higher compared the mass observed one-day post seeding.

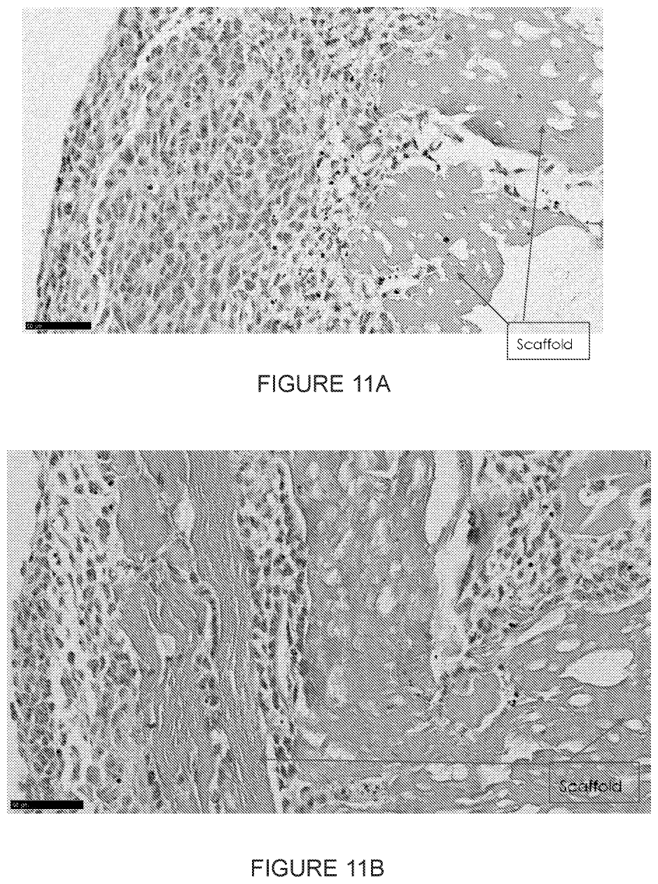

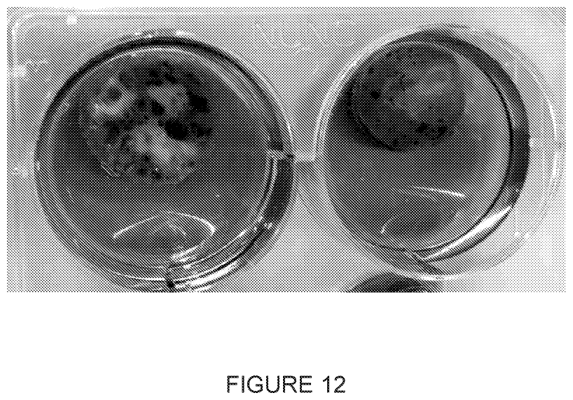

FIG. 11 further shows that the cells are functional, as PSR staining shows the production of collagen as the hallmark protein produced by fibroblasts. Collagen contributes to the meat cut nutritional and sensorial properties.

### Example 7: Cell Differentiation on the Edible Products of the Invention Used as Scaffold

The edible product G70 prepared by covered steaming (G70CS) or by covered baking (G70CB) was examined for its suitability for use as a scaffold for culturing cells. G70CS and G70CB sections (20 mm diameter, 3 mm thick) were vacuumed and sterilized by Gamma irradiation (25-40 KGy, by Sor-van, Israel), then seeded with cells.

Bovine Pluripotent Stem Cells (bPSCs) that were differentiated to bovine Stromal Progenitor Cells (bSPCs) in a Stirred Tank Bioreactor (STB) for 5 days were seeded on the sections (G70CB and G70CS) and incubated for 12 days for further differentiation to muscle and collagen producing cells.

The differentiation of bovine Stromal Progenitor Cells (bSPCs) to Muscle and Collagen producing cells on the G70CB and G70CS sections was evaluated by qPCR analysis of muscle or collagen marker genes (MyHC3 and COLIII, respectively), as presented in Tables 7-8 below. β-2 microglobulin (B2M) was used as endogenous control (constant expressed reference gene). The relative quantification (RQ) level of each gene was calculated relative to the expression in Bovine Pluripotent Stem Cells (bPSCs) using the following equation:

RQ(tested gene)=2^{-{ΔCT(tested gene in tested sample)-ΔCT(tested gene in bPSC)}}

Where:
CT—cycle threshold, is defined as the number of cycles required for the fluorescent signal to cross a predetermined threshold.
ΔCT(tested gene in tested sample)=CT(tested gene in tested sample)−CT(B2M gene in tested sample)
ΔCT(tested gene in bPSC)=CT(tested gene in bPSC)−CT(B2M gene in bPSC)

The expression of COL III in Bovine Embryo Fibroblast (BEF) was used as a positive control.

TABLE 7
Muscle and Collagen gene expression by
bovine cells cultured on G70CS scaffold

| | Marker | | | | |
|---|---|---|---|---|---|
| | B2M | COL III | | MyHC3 | |
| Sample description | CT | CT | RQ | CT | RQ |
| bPSC | 21.47 | 30.30 | 1 | 32.86 | 1 |
| bSPC | 23.00 | 28.11 | 13 | 33.73 | 2 |
| Final cultivation of | 25.84 | 20.89 | 14073 | 31.59 | 50 |
| bSPC seeded on G70CS | | | | | |
| BEF | 23.02 | 18.98 | 7490 | 29.78 | 25 |

TABLE 8
Muscle and Collagen gene expression by
bovine cells cultured on G70CB scaffold

| | Marker | | | | |
|---|---|---|---|---|---|
| | B2M | COL III | | MyHC3 | |
| Sample description | CT | CT | RQ | CT | RQ |
| bPSC | 21.47 | 30.30 | 1 | 32.86 | 1 |
| bSPC | 23.00 | 28.11 | 13 | 33.73 | 2 |
| Final cultivation of | 26.10 | 21.49 | 11118 | 22.76 | 27048 |
| bSPC seeded on G70CB | | | | | |
| BEF | 23.02 | 18.98 | 7490 | 29.78 | 25 |

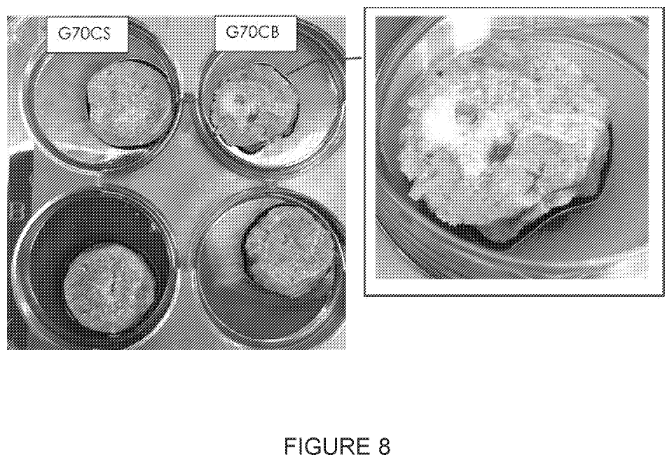

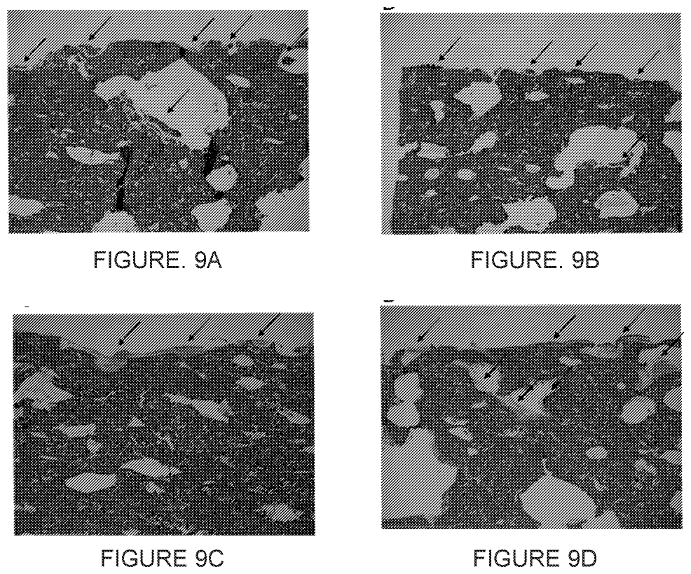

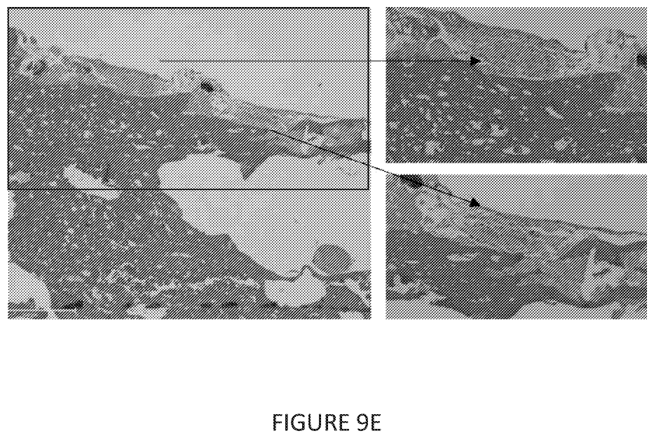

Samples with cell culture, end of growing phase, are shown in FIG. 8. At the end on the differentiation stage, cells were stained by H & E staining. FIG. 9 shows G70CS and G70CB with cells. The image with cells shows morphology of the differentiated cells (small black dots are the nuclei).

As can be seen in Tables 7 and 8, bSPCs seeded on either G70CS or G70CB presented high expression of COLIII indicating that the cells were differentiated to collagen producing cells. Cells seeded on G70CB presented also high expression of MyHC3, indicating that the cells were differentiated also to muscle cells. These results suggest that the preparation procedure of the scaffolds can affect the differentiation patterns of bSPC.

### Example 8: Differentiation of Bovine Embryonic Stem Cells to Adipocytes on a Meatloaf Scaffold

The edible porous product, (baked or steamed) shaped as a scaffold in diameter of 20 mm and width of 3 mm, was placed in a tissue culture plate and pre-washed using DMEMF12. Bovine embryonic fibroblasts (BEFs) were seeded on the scaffold (8×10⁶ cells/cm²) in minimal volume of serum-free growth medium (30×10⁶ cells/150 μl i.e., 200,000 cells/μl) and were incubated at 38.5° C. with 5% CO₂ for 24 h.

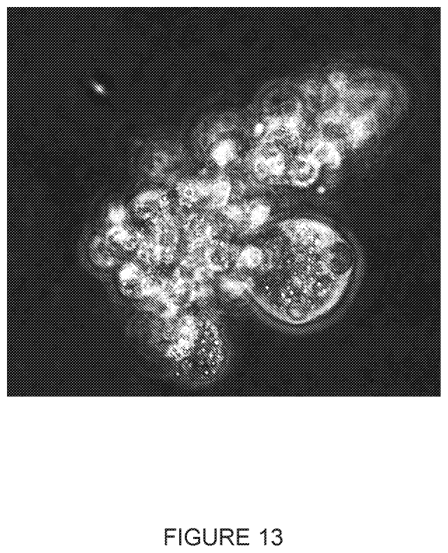

During these 24 h the cells were slightly attached to the scaffold that provided a soft matrix that induced conditions supporting adipogenesis. After 24 h, the medium was refreshed with standard adipogenic differentiation medium that was added to the well, covering the scaffold (12). After total of 8 days of growth and differentiation, the cells were washed out of the scaffold and analyzed for adipocyte morphology using light microscope (FIG. 13).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An edible product comprising a protein combination and water and having a porous structure, wherein the protein combination comprises at least 40% (w/w) Triticeae gluten out of the total amount of said protein combination and at least one additional type of non-Triticeae protein, and wherein the porous product is non-extruded.

2. (canceled)

3. The edible product of claim 1, wherein the Triticeae plant is a Triticum plant selected from the group consisting of: Triticum aestivum (bread wheat) and Triticum spelta (Spelt).

4. (canceled)

5. The edible product of claim 1, wherein the at least one additional protein is derived from at least one of a plant other than a Triticeae plant, a fungus, an alga, a single cell microorganism and any combination thereof.

6. (canceled)

7. The edible product of claim 1, wherein the amount of the protein combination within the edible porous product is from about 10% to about 50% w/w out of the total product weight.

8. (canceled)

9. (canceled)

10. The edible product of claim 1, wherein the edible product further comprises at least one of an aqueous soluble salt, edible oil, edible fat, and any combination thereof.

11. (canceled)

12. The edible product of claim 1, wherein the edible porous product further comprises at least one agent selected from the group consisting of: a flavoring agent precursor, a flavoring agent, a hydrocolloid, a gelling agent, a thickening agent, an emulsifying agent, a binder, a stabilizer, an agent promoting protein-protein bonding, an agent enhancing cell adherence to the edible porous product, an anti-oxidizing agent, a coloring agent and any combination thereof.

13. (canceled)

14. The edible product of claim 1, wherein at least 35% of the total volume of the edible porous product is void volume.

15-18. (canceled)

19. The edible product of claim 1, wherein said edible product is for use as a scaffold for culturing at least one type of cells.

20. The edible product of claim 19, wherein the cultured cells are non-human-animal cells.

21. The edible product of claim 20, wherein said edible product is a cultured meat product.

22. A food product comprising an edible porous product comprising a combination of proteins and water, wherein the combination of protein comprises at least 40% Triticeae gluten out of the total weight of said combination and at least one additional type of non-Triticeae protein; and at least one type of non-human-animal cells.

23-27. (canceled)

28. The food product of claim 22, wherein the at least one type of non-human-animal cells is cultured on and/or within the edible porous product.

29. The food product of claim 22, wherein the at least one type of non-human-animal cells is mixed with the edible porous products or parts thereof.

30-32. (canceled)

33. A method for producing an edible porous product comprising the steps of:
a) blending a combination of components comprising a proteins combination and water at a ratio of from about 1:1 to about 1:3 protein combination to water, wherein the protein combination comprises at least 40% w/w Triticeae gluten out of the total amount of the protein combination and at least one additional type of non-Triticeae protein to form a malleable material; and
b) exposing the malleable material to thermal treatment at a temperature of from about to about 250° C. at a pressure up to an atmospheric pressure;
thereby producing the edible porous product, wherein the edible product is characterized by a porous structure.

34-36. (canceled)

37. The method of claim 33, wherein the blending step comprises:
a) mixing the protein combination and potentially at least one additional ingredient to obtain a homogenous blend of dry components;
b) adding water and optionally at least one additional liquid to form a mixture;
c) kneading or mixing the mixture of dry components, water and optionally at least one additional liquid to form homogenous dough-like material; and
d) kneading or mixing the dough-like material of step (c) until the dough-like material develops gluten network and porous structure.

38-42. (canceled)

43. The method of claim 33, wherein said method further comprising a step of seeding at least one type of non-human-animal cells on and/or within at least part of the edible porous product and culturing the seeded edible porous product under condition enabling the formation of a tissue, thereby producing an edible porous product comprising cultured cells and/or tissues thereon and/or therewithin.

44. The method of claim 33, wherein said method further comprising mixing at least one type of non-human-animal cells with the edible porous product thereby producing an edible hybrid protein-meat product.

45-49. (canceled)

50. A method for producing a plurality of adipocytes, comprising culturing a plurality of mesoderm-derived non-human-animal cells on and/or within an edible porous product comprising a combination of proteins and water, wherein the combination of protein comprises at least 40% Triticeae gluten out of the total weight of said combination and at least one additional type of non-Triticeae protein.

51. The method of claim 50, wherein culturing the plurality of mesoderm-derived non-human animal-cells comprises furnishing said plurality of cells with a cell culture medium.

52. The method of claim 50, wherein the non-human-animal cells are bovine cells, thereby producing bovine adipocytes.
